(19) United States
(12) Patent Application Publication
(10) Pub. No.: US 2026/0256471 A1
(43) Pub. Date: Sep. 3, 2026

(54) STAPLE CARTRIDGE WITH KNIFE

(71) Applicant: Covidien LP, Mansfield, MA (US)

(72) Inventors: Logamurugaraj SHANMUGAVEL, Hyderabad (IN); Suresh Kumar PREMA MOHANASUNDARAM, North Haven, CT (US); Hari Naga Mahesh KALEPU, Hyderabad (IN); Satish GUNDALA, Hyderabad (IN)

(73) Assignee: Covidien LP, Mansfield, MA (US)

(21) Appl. No.: 18/877,759

(22) PCT Filed: Jun. 19, 2023
(86) PCT No.: PCT/IB2023/056323

§ 371 Date: Dec. 20, 2024

Related U.S. Application Data
(60) Provisional application No. 63/354,770, filed on Jun. 23, 2022.

(51) Int. Cl. A61B 17/072; A61B 17/00

(52) U.S. Cl. CPC A61B 17/07207; A61B 2017/00862; A61B 2017/07271; A61B 2017/07278; A61B 2017/07285

(57) ABSTRACT

A surgical stapling device includes a drive assembly and tool assembly having an anvil assembly and a cartridge assembly. The cartridge assembly has a staple cartridge including a cartridge body, staples, pushers, and an actuation sled assembly. The actuation sled assembly includes a knife. The actuation sled assembly and the drive assembly are configured to interact to allow the knife to be in exposed and unexposed conditions.

### FIELD

The disclosure is directed to surgical stapling devices and, more particularly, to a surgical stapling device having a replaceable staple cartridge having a knife.

### BACKGROUND

Surgical stapling devices for simultaneously stapling and cutting tissue are well known in the art and include a tool assembly and a drive assembly. The tool assembly includes an anvil assembly and a cartridge assembly that are movable in relation to each other between open and clamped positions. The cartridge assembly has a channel member and a staple cartridge that is received within the channel member. Typically, the staple cartridge includes a cartridge body that supports staples, pushers, and an actuation sled, and the actuation sled is movable through the cartridge body from a retracted position to an advanced position into sequential engagement with the pushers to sequentially eject the staples from the cartridge body. The staple cartridge may be releasably received within the channel member such that it can be replaced to facilitate reuse of the stapling device.

Typically, the drive assembly supports a knife and is movable from a retracted position to an advanced position through the tool assembly to advance the actuation sled through the tool assembly. When the drive assembly moves from the retracted position to the advanced position, the knife moves through the tool assembly to cut tissue. When the staple cartridge is replaced after each firing of the stapling device, the drive assembly is reused and the knife may be dulled with each reuse of the stapling device. Staple cartridges having knives are known in the art. In such devices, the knife must be shielded after each use and prior to replacement to prevent injury to a clinician during staple cartridge replacement.

A continuing need exists for a staple cartridge having a knife that includes structure that can adequately and reliably shield the knife during staple cartridge replacement.

### SUMMARY

This disclosure is directed to a surgical stapling device that includes a drive assembly and tool assembly having an anvil assembly and a cartridge assembly. The cartridge assembly has a staple cartridge including a cartridge body, staples, pushers, and an actuation sled assembly. The actuation sled assembly includes a knife. The actuation sled assembly and the drive assembly are configured to interact to allow the knife to be in exposed and unexposed conditions.

Aspects of the disclosure are directed to a surgical stapling device including an elongate body, a tool assembly, and a drive assembly. The elongate body has a proximal portion and a distal portion. The tool assembly is supported on the distal portion of the elongate body and includes an anvil assembly and a cartridge assembly. The cartridge assembly is coupled to the anvil assembly to facilitate movement of the tool assembly between open and clamped positions. The cartridge assembly includes a channel member and a staple cartridge. The channel member defines a channel, and the staple cartridge is supported within the channel. The staple cartridge includes a cartridge body, staples, pushers, and an actuation sled assembly. The cartridge body has a tissue engaging surface and defines a knife slot and staple receiving slots positioned on each side of the knife slot. The staples are received in the staple receiving slots. The actuation sled assembly is movable within the cartridge body from a sled retracted position to a sled advanced position to eject the staples from the staple receiving slots of the cartridge body. The actuation sled assembly includes an actuation sled, a knife, and a biasing member. The knife is received within the knife slot of the cartridge body and includes a cutting edge. The knife is secured to the actuation sled and is movable from a lowered position in which the cutting edge is shielded within the knife slot of the cartridge body to a raised position in which the cutting edge of the knife extends from the knife slot above the tissue engaging surface of the cartridge body. The biasing member is secured to the actuation sled and positioned to urge the knife to the lowered position. The drive assembly includes a working member that is positioned proximally of the actuation sled assembly and movable into engagement with the knife of the actuation sled assembly to move the knife from the lowered position to the raised position.

Other aspects of the disclosure are directed to a cartridge assembly including a channel member and a staple cartridge. The channel member defines a channel. The staple cartridge is supported within the channel of the channel member and includes a cartridge body, staples, pushers, and an actuation sled assembly. The cartridge body has a tissue engaging surface and defines a knife slot and staple receiving slots positioned on each side of the knife slot. The staples are received in the staple receiving slots. The actuation sled assembly is movable within the cartridge body from a sled retracted position to a sled advanced position to eject the staples from the staple receiving slots of the cartridge body. The actuation sled assembly includes an actuation sled, a knife, and a biasing member. The knife is received within the knife slot of the cartridge body and includes a cutting edge. The knife secured to the actuation sled and is movable from a lowered position in which the cutting edge is shielded within the knife slot of the cartridge body to a raised position in which the cutting edge of the knife extends from the knife slot above the tissue engaging surface of the cartridge body. The biasing member is secured to the actuation sled and positioned to urge the knife to the lowered position.

In aspects of the disclosure, the actuation sled includes a central portion that is received in the knife slot of the cartridge body and defines a recess that receives the knife.

In some aspects of the disclosure, the knife includes a base portion having a circular configuration that is rotatably received within the recess in the central portion of the actuation sled.

In certain aspects of the disclosure, that biasing member is secured to the central portion of the actuation sled and includes a resilient finger that engages the base portion of the knife to urge the knife towards the lowered position.

In aspects of the disclosure, the biasing member is secured to the central portion of the actuation sled and includes a torsion spring that is engaged with the knife to urge the knife towards the lowered position.

In some aspects of the disclosure, the knife includes a curved proximally facing surface and the working end of the drive assembly includes a distally facing curved cam surface.

In certain aspects of the disclosure, the distally facing curved cam surface of the working end of the drive assembly is movable into the curved proximally facing surface of the knife to move the knife from the lowered position to the raised position.

In aspects of the disclosure, the biasing member includes an elastic band.

In some aspects of the disclosure, the knife is movable vertically within the recess between the lowered position and the raised position.

In certain aspects of the disclosure, the elastic band has a first end secured to the knife and a second end secured to the actuation sled.

In aspects of the disclosure, the knife has a proximally facing angled surface and the working member of the drive assembly includes an angled cam surface.

In some aspects of the disclosure, the angled cam surface of the working member of the drive assembly is movable into engagement with the proximally facing angled surface of the knife to move the knife from the lowered position to the raised position.

Other aspects of the disclosure are directed to cartridge assembly including a channel member and a staple cartridge. The channel member defines a channel the staple cartridge is supported within the channel. The staple cartridge includes a cartridge body, staples, pushers, and an actuation sled assembly. The cartridge body has a tissue engaging surface and defines a knife slot and staple receiving slots positioned on each side of the knife slot. The staples are received in the staple receiving slots. The actuation sled assembly is movable within the cartridge body from a sled retracted position to a sled advanced position to eject the staples from the staple receiving slots of the cartridge body. The actuation sled assembly includes an actuation sled, a knife, a cover, and a biasing member. The knife is secured to the actuation sled and received within the knife slot of the cartridge body. The knife has a cutting edge. The cover is pivotally secured to the knife and defines a pocket. The cover is movable from a first position in which the cutting edge of the knife is received within the pocket of the cover and a second position in which the cutting edge of the knife is exposed. The biasing member is secured to the knife and positioned to urge the cover towards the first position.

In aspects of the disclosure, the biasing member includes a spring arm, and the cover includes a finger, and the spring arm is engaged with the finger of the cover to urge the cover towards the first position.

Other features of the disclosure will be appreciated from the following description.

### BRIEF DESCRIPTION OF THE DRAWINGS

Various aspects of the disclosure are described herein below with reference to the drawings, wherein:

FIG. 1 is side perspective view of a stapling device according to aspects of the disclosure including a tool assembly in an open position;

FIG. 2 is a side perspective view of the tool assembly shown in FIG. 1 with the staple cartridge removed from a channel member of a cartridge assembly of the tool assembly;

FIG. 3 is an exploded view of the staple cartridge of the tool assembly shown in FIG. 2;

FIG. 4 is an enlarged view of the indicated area of detail shown in FIG. 3 illustrating an actuation sled assembly of the staple cartridge;

FIG. 5 is an exploded view of the actuation sled assembly shown in FIG. 4;

FIG. 6 is an enlarged view of the indicated area of detail shown in FIG. 2;

FIG. 7 is a side perspective view of the tool assembly of the stapling device shown in FIG. 1 showing the actuation sled assembly and a drive assembly with the remaining components shown in phantom;

FIG. 8 is a cross-sectional view taken along section line 8-8 of FIG. 7;

FIG. 9 is a side cross-sectional view taken through the tool assembly shown in FIG. 7 as the drive assembly and the actuation sled assembly move from retracted positions towards advanced positions;

FIG. 10 is a side cross-sectional view taken through the tool assembly shown in FIG. 7 with the drive assembly and the actuation sled assembly in advanced positions;

FIG. 11 is a side cross-sectional view taken through the tool assembly shown in FIG. 7 as the drive assembly and the actuation sled assembly move from the advanced positions back towards the retracted positions;

FIG. 12 is a side perspective view of an alternate version of the drive assembly and the actuation sled assembly of the stapling device shown in FIG. 1 with the drive assembly spaced from the actuation sled assembly;

FIG. 13 is a cross-sectional view taken along section line 13-13 of FIG. 12;

FIG. 14 is a cross-sectional view of a distal portion of the drive assembly and the actuation sled assembly shown in FIG. 12 with the drive assembly engaged with the actuation sled assembly;

FIG. 15 is a side perspective exploded view of an alternate version of the actuation sled assembly of the surgical stapling device shown in FIG. 1;

FIG. 16 is a side cross-sectional view taken through the actuation sled assembly shown in FIG. 15 and an alternate version of a drive assembly of the stapling device shown in FIG. 1 with the drive assembly spaced from the actuation sled assembly;

FIG. 17 is a side cross-sectional view taken through the actuation sled assembly and the drive assembly shown in FIG. 16 with the distal portion of the drive assembly engaged with the actuation sled assembly;

FIG. 18 is a side perspective an alternate version of the actuation sled assembly of the surgical stapling device shown in FIG. 1;

FIG. 19 is a side perspective exploded view of the actuation sled assembly shown in FIG. 18;

FIG. 20 is a side cross-sectional view taken through the actuation sled assembly shown in FIG. 19 and an alternate version of a drive assembly of the stapling device shown in FIG. 1 with the drive assembly spaced from the actuation sled assembly;

FIG. 21 is a cross-sectional view taken along section line 21-21 of FIG. 20; and

FIG. 22 is a cross-sectional view taken through the actuation sled assembly and drive assembly shown in FIG. 20 with the distal portion of the drive assembly engaged with the actuation sled assembly.

### DETAILED DESCRIPTION

The disclosed surgical stapling device will now be described in detail with reference to the drawings in which like reference numerals designate identical or corresponding elements in each of the several views. However, it is to be understood that aspects of the disclosure are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the stapling device in its customary manner, while the term “distal” is used generally to refer to that portion of the device that is farther from the clinician during use of the stapling device in its customary manner. In addition, the term “clinician” is used generally to refer to medical personnel including doctors, nurses, and support personnel. Further, directional terms such as front, rear, upper, lower, top, bottom, and similar terms are used to assist in understanding the description and are not intended to limit the disclosure.

The disclosed surgical stapling device includes a tool assembly having an anvil assembly, a cartridge assembly, and a drive assembly. The cartridge assembly has a channel member that defines a channel and a staple cartridge that is received within the channel. The staple cartridge includes a cartridge body, staples, pushers, and an actuation sled assembly. The drive assembly has a working member that is movable through the tool assembly to advance the actuation sled assembly through the staple cartridge to actuate the tool assembly. The actuation sled assembly includes an actuation sled, a knife, and a biasing member. The knife is movable in relation to the actuation sled between raised and lowered positions, and the biasing member is positioned to move the knife towards the lowered position. The drive assembly is movable into engagement with the actuation sled assembly to move the knife from the lowered position to the raised position.

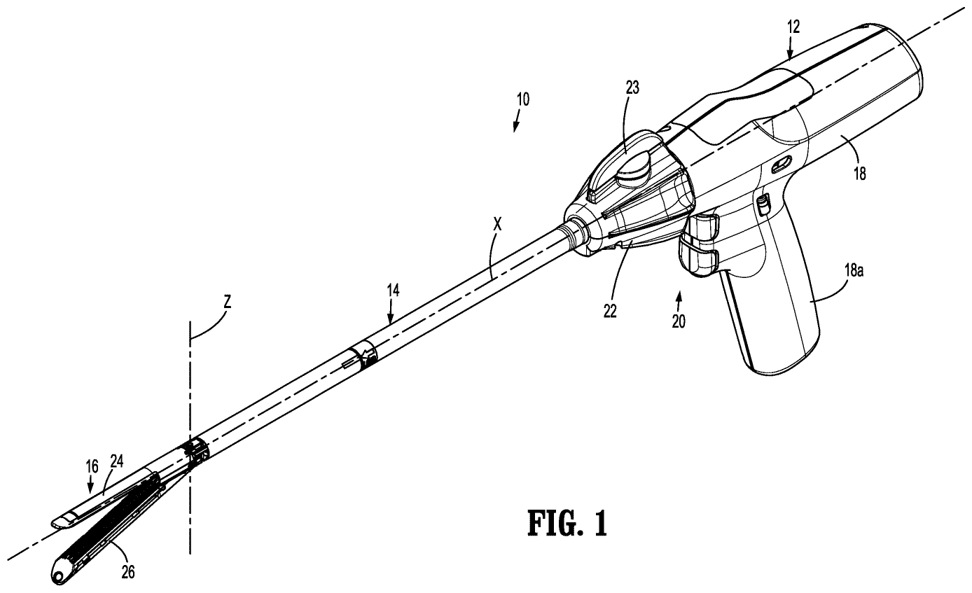

FIG. 1 illustrates a surgical stapling device according to aspects of the disclosure shown generally as stapling device 10. The stapling device 10 includes a handle assembly 12, an elongate body 14, and a tool assembly 16. The elongate body 14 defines a longitudinal axis “X” (FIG. 1). The handle assembly 12 includes a body 18 that defines a hand grip 18a, a plurality of actuator buttons 20, and a rotation knob 22. The rotation knob 22 is rotatably supported on a distal portion of the body 18 of the handle assembly 12 and supports the elongate body 14 to facilitate rotation of the elongate body 14 and the tool assembly 16 about the longitudinal axis “X” of the elongate body 14 in relation to the handle assembly 12. The actuator buttons 20 control operation of the various functions of the stapling device 10 including articulation, clamping, firing, and cutting of tissue. In aspects of the disclosure, the rotation knob supports an articulation lever 23 that forms part of an articulation mechanism that is operable to pivot the tool assembly 16 about an articulation axis “Z” that is perpendicular to the longitudinal axis “X”.

The tool assembly 16 includes an anvil assembly 24 and a cartridge assembly 26 that are movable in relation to each other between an open position (FIG. 1) and a clamped position. In aspects of the disclosure, the anvil assembly 24 is fixed in relation to the elongate body 14 and the cartridge assembly 26 is pivotable towards and away from the anvil assembly 24. However, it is envisioned that the cartridge assembly 26 could be fixed in relation to the elongate body 14 and the anvil assembly 24 could pivot towards and away from the cartridge assembly 26. Although the stapling device 10 is illustrated as an electrically powered stapling device, it is envisioned that the tool assembly 16 would also be suitable for use with manually powered surgical stapling devices and robotically operated stapling systems. U.S. Pat. No. 9,055,943 discloses a surgical stapling device including a powered handle assembly, U.S. Pat. No. 6,241,139 discloses a surgical stapling device including a manually actuated handle assembly, and U.S. Pat. No. 9,962,159 discloses a stapling device that is configured for use with a robotic system.

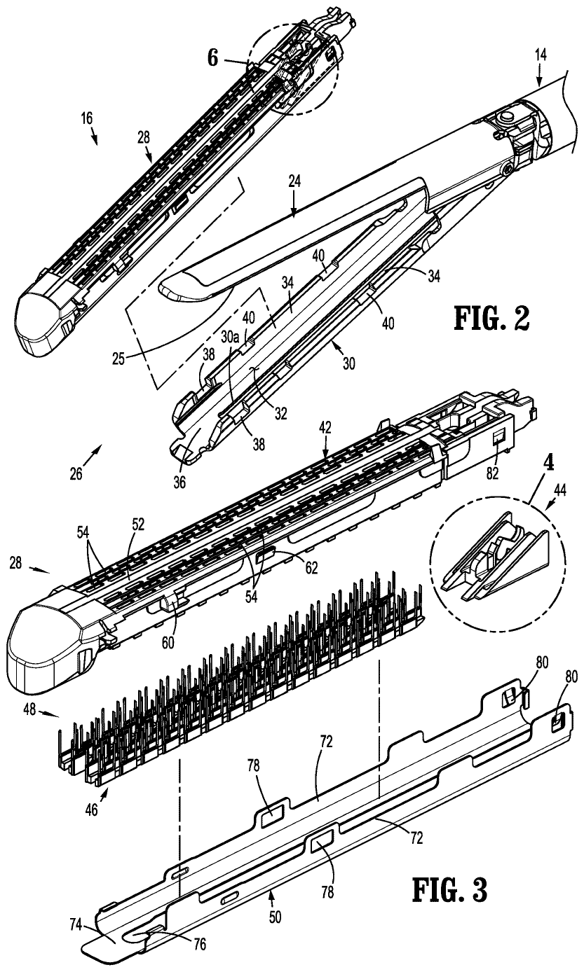

FIG. 2 illustrates the tool assembly 16 which includes the anvil assembly 24 and the cartridge assembly 26. The anvil assembly 24 includes a staple forming surface 25. The cartridge assembly 26 includes a staple cartridge 28 and a channel member 30 that defines a channel 32 that receives the staple cartridge 28. In aspects of the disclosure, the staple cartridge 28 is releasably secured within the channel 32 to facilitate reuse of the stapling device 10. The channel member 30 includes side walls 34 and a bottom wall 36 that define the channel 32. The bottom wall 36 defines a knife slot 30a. In aspects of the disclosure, the side walls 34 of the channel member 30 define cutouts 38 and notches 40 that are provided to align and retain the staple cartridge 28 within the channel 32 of the channel member 30 as described in further detail below.

FIG. 3 illustrates the staple cartridge 28 which includes a cartridge body 42, an actuation sled assembly 44, pushers 46, staples 48, and a base plate 50. The cartridge body 42 defines a knife slot 52 and staple receiving slots 54 positioned on opposite sides of the knife slot 52. The knife slot 52 in the cartridge body 42 is longitudinally aligned with the knife slot 30a of the channel member 30 when the staple cartridge 28 is received in the channel 32 of the channel member 30. In aspects of the disclosure, the staple receiving slots 54 are aligned in rows on each side of the knife slot 52. Although three rows of staple receiving slots 54 are shown on each side of the knife slot 52, it is envisioned that the cartridge body 42 may define one or more rows of staple receiving slots 54 on each side of the knife slot 52.

The pushers 46 support the staples 48 within each of the staple receiving slots 54 of the cartridge body 42. The actuation sled assembly 44 is movable within the cartridge body 42 from a sled retracted position to a sled advanced position and is positioned to sequentially engage the pushers 46 to eject the staples 48 from the cartridge body 42. The base plate 50 is secured to a bottom of the cartridge body 42 to prevent the pushers 46 and the staples 48 from falling from the cartridge body 42.

In aspects of the disclosure, the cartridge body 42 includes outwardly extending protrusions 60 that are received within the cutouts 38 in the side walls 34 of the channel member 30 to properly position the staple cartridge 28 within the channel 32 of the channel member 30. In certain aspects of the disclosure, the cartridge body 42 also includes tabs 62 that are received in the notches 40 in the side walls 34 of the channel member 30 to releasably retain the staple cartridge within the channel 32 of the channel member 30.

The base plate 50 of the staple cartridge 28 includes side walls 72 and a bottom wall 74 that define a channel that receives the cartridge body 42 of the staple cartridge 28. The bottom wall 74 of the base plate 50 defines a knife slot 76 that is longitudinally aligned with the knife slots 52 and 30a of the cartridge body 42 and the channel member 30, respectively. The side walls 72 of the base plate 50 define openings 78 that receive the tabs 62 of the cartridge body 42 in snap-fit fashion to secure the base plate 50 to the cartridge body 42. In aspects of the disclosure, the proximal portion of the side walls 72 of the base plate 50 also includes inwardly extending resilient fingers 80 that snap into notches 82 in the cartridge body 42 to secure the base plate 50 to the cartridge body.

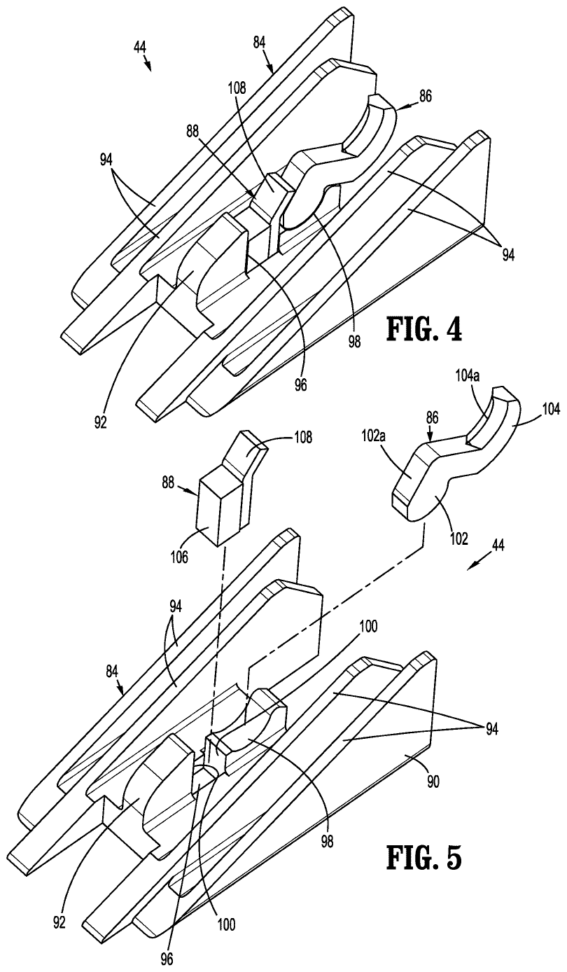

FIGS. 4-7 illustrate the actuation sled assembly 44 which includes an actuation sled 84, a knife 86, and a biasing member 88. The actuation sled 84 includes a sled body 90 that has a central portion 92 and cam surfaces 94 that are positioned on opposite sides of the central portion 92 of the sled body 90. The cam surfaces 94 define ramps that are positioned to engage the pushers 46 (FIG. 3) as the actuation sled 84 moves from the sled retracted position towards the sled advanced position to sequentially eject the staples 48 (FIG. 3) from the cartridge body 42 of the staple cartridge 28. The central portion 92 of the sled body 90 of the actuation sled 84 is received within the knife slot 52 of the cartridge body 42 and defines a slot 96 and a curved or semi-circular recess 98. In aspects of the disclosure, the recess 98 is defined by a wall of the central body 92 of the actuation sled having a curved or semi-circular configuration and the slot 96 is defined by longitudinally spaced planar surfaces 100.

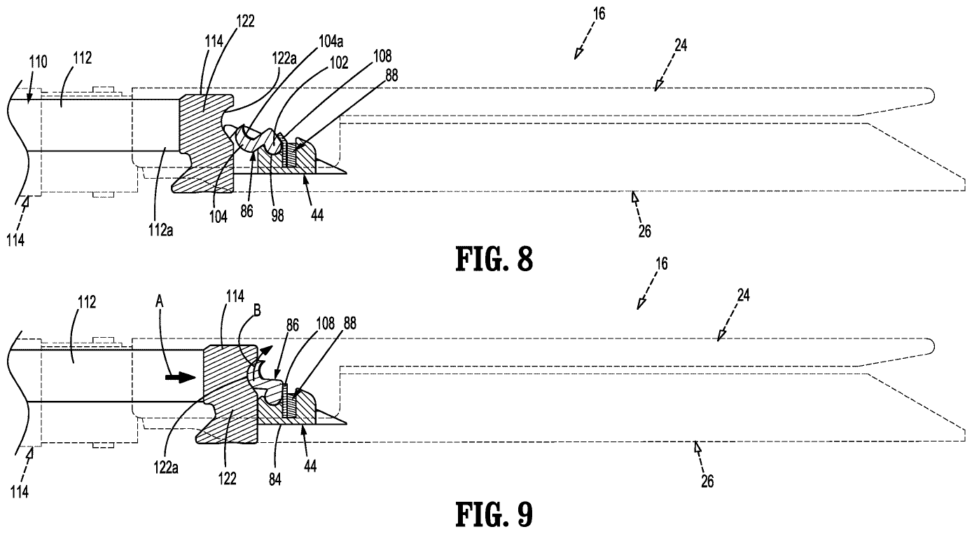

The knife 86 includes a base portion 102 that is rotatably received in the curved recess 98 of the central portion 92 of the actuation sled 90 such that the knife 86 is received within the knife slot 52 of the cartridge body 42. In aspects of the disclosure, the base portion 102 of the knife 86 has a circular configuration. The knife 86 includes a knife portion 104 that extends upwardly from the base portion 102 and includes a distally facing cutting edge 104a. In aspects of the disclosure, the knife portion 104 includes a curved proximally facing surface. The knife 86 is rotatable in the recess 98 of the central portion 92 of the actuation sled 90 such that the cutting edge 104a of the knife 86 is movable between a first position located within the knife slot 52 of the cartridge body 42 and a second position extending above the knife slot 52 of the cartridge body 42 towards the anvil assembly 24. In the first position of the knife 86 (FIG. 8), the curved knife portion 104 of the knife 86 extends proximally of the actuation sled 84.

The biasing member 88 includes a body portion 106 and a proximally extending resilient finger 108. The body portion 106 of the biasing member 88 is received and secured within the slot 96 of the central portion 92 of the sled body 90 such that the finger 108 of the biasing member 88 is engaged with the base portion 102 of the knife 86. In aspects of the disclosure, the finger 108 is angled towards the base portion 102 of the knife 86, and the base portion 102 of the knife 86 includes an angled surface 102a that is engaged with the finger 108. The finger 108 is configured to urge the knife to the lowered position.

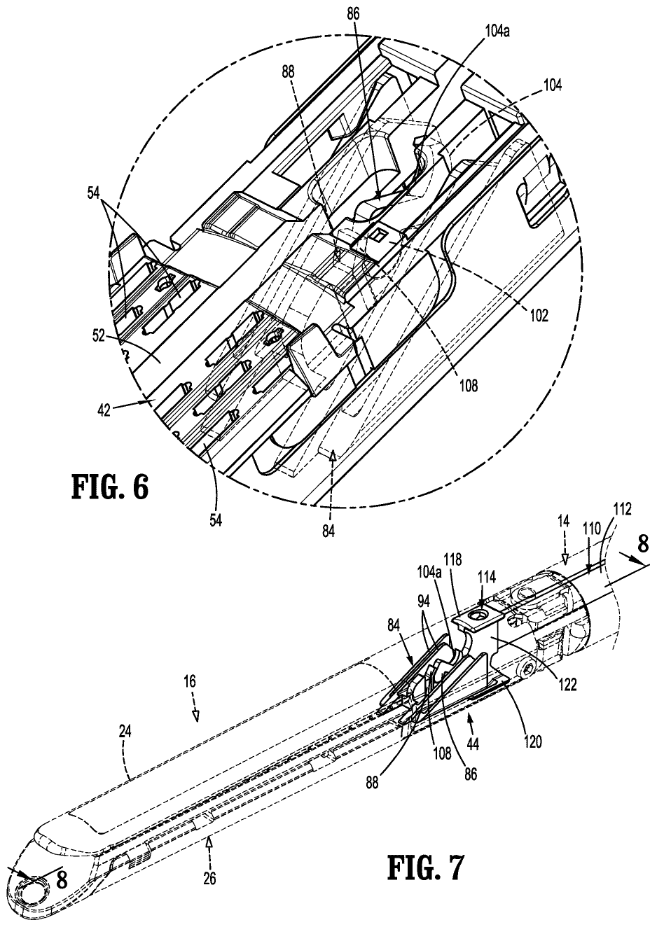

FIGS. 6-8 illustrate a drive assembly 110 of the stapling device 10 (FIG. 1). The drive assembly 110 includes a resilient and/or flexible drive beam 112 and a working member 114. The drive beam 112 has a proximal portion (not shown) that is coupled to a drive rod (not shown) supported within the elongate body 14 of the stapling device 10 (FIG. 1) and a distal portion 112a that is coupled to the working member 114. In aspects of the disclosure, the resilient drive beam 112 is formed from laminated sheets of material, e.g., steel, that are welded to the working member 114. Alternately other materials of construction and securement techniques are envisioned.

The working member 114 of the drive assembly 110 has an I-beam configuration and includes a first beam 118, a second beam 120, and a vertical strut 122 that connects the first beam 118 to the second beam 120. The working member 114 is positioned proximally of the actuation sled assembly 44 (FIG. 4) and is movable, in response to activation of the actuation buttons 20 (FIG. 1), through the tool assembly 16 between retracted and advanced positions to move the actuation sled assembly 44 through the tool assembly 16 (FIG. 1) from the sled retracted position to the sled advanced position. The vertical strut 122 has a distally facing curved cam surface 122a (FIG. 8) that is in partial alignment with the curved proximally facing surface of the knife portion 104 of the knife 86.

FIG. 8 illustrates the distal portion of the stapling device 10 (FIG. 1) including the tool assembly 16 with the tool assembly 16 in the clamped position. When the tool assembly 16 is in the clamped position, the working member 114 of the drive assembly 110 is spaced from the actuation sled assembly 44 such that the knife 86 of the actuation sled assembly 44 is urged to the lowered position by the finger 108 of the biasing member 88. In the lowered position, the cutting edge 104a of the knife 86 is recessed within the knife slot 52 of the cartridge body 42 and is shielded. As illustrated, the knife portion 104 of the knife 86 is partially aligned with the distally facing cam surface 122a of the working member 114 of the drive assembly 86.

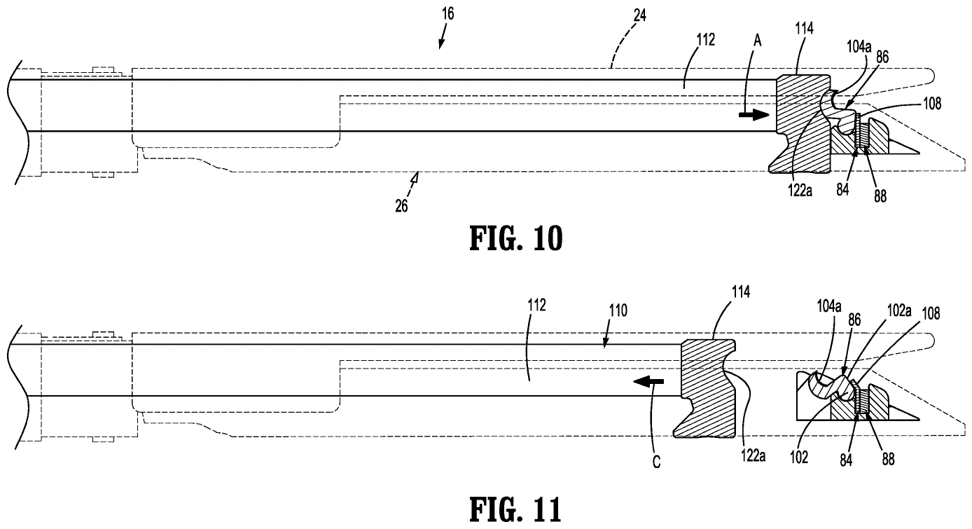

FIG. 9 and illustrate the tool assembly 16 of the stapling device 10 (FIG. 1) as the drive assembly 110 is advanced in the direction of arrow “A” into engagement with the actuation sled assembly 44 to fire the stapling device 10. When the drive assembly 110 is advanced to fire the stapling device 10 (FIG. 1), the working member 114 moves into engagement with knife 86 of the actuation sled assembly 44 to move the knife 86 from the lowered position to the raised position. More specifically, when the working member 114 of the drive assembly engages the knife 86 of the actuation sled assembly 44, the curved proximally facing surface of the knife portion 104 of the knife 86 is received within the distally facing cam surface 122a of the vertical strut 122 of the working member 114 and is cammed upwardly in the direction of arrow “B” against the urging of the finger 108 of the biasing member 88 which is deformed. As the knife is pivoted to the raised position, the base portion 102 of the knife 86 rotates within the recess 98 of the central portion 92 of the actuation sled 84. In the raised position, the cutting edge 104a of the knife 86 extends upwardly from the knife slot 152 of the cartridge body 42 of the staple cartridge 28 (FIG. 10) through a knife slot (not shown) in the anvil assembly 24 (FIG. 10). As the drive assembly 110 is moved to its advanced position, the actuation sled assembly 44 moves through the cartridge body 42 and the knife 86 remains in the raised position (FIG. 10).

FIG. 11 illustrates the tool assembly 16 of the stapling device 10 (FIG. 1) as the drive assembly 110 is retracted from the advanced position towards the retracted position in the direction of arrow “C”. The working member 114 of the drive assembly 110 is in abutting relation to the actuation sled assembly 44 but is not coupled to the actuation sled assembly 44. When the working member 114 of the drive assembly 110 is moved from the advanced position back towards the retracted position, the working member 114 moves away from the knife 86. When the working member 114 moves away from the actuation sled assembly 44, the finger 108 of the biasing member 88 returns to its non-deformed configuration and urges the knife 86 from the raised position to the lowered position. As described above, in the lowered position, the cutting edge 104a of the knife 86 is recessed and shielded within the cartridge body 42.

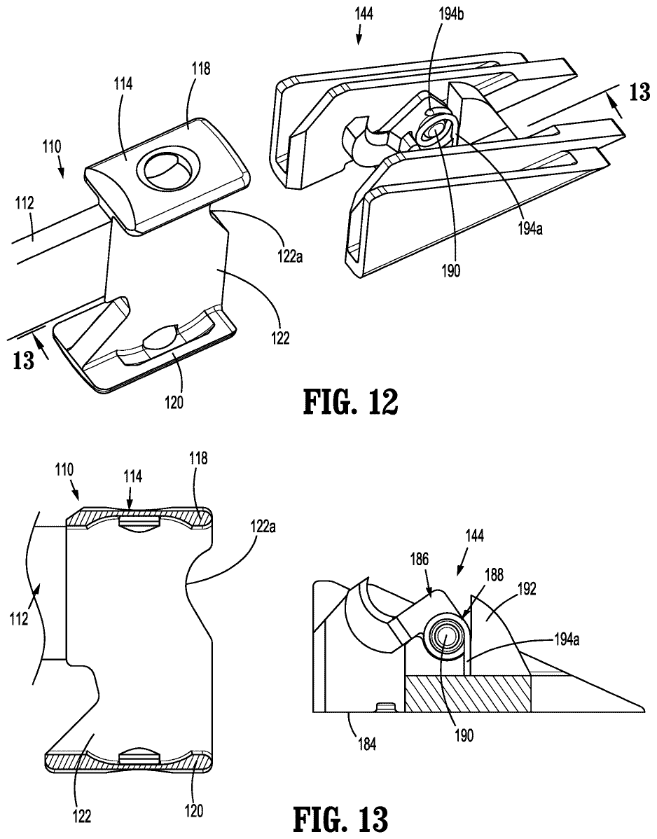

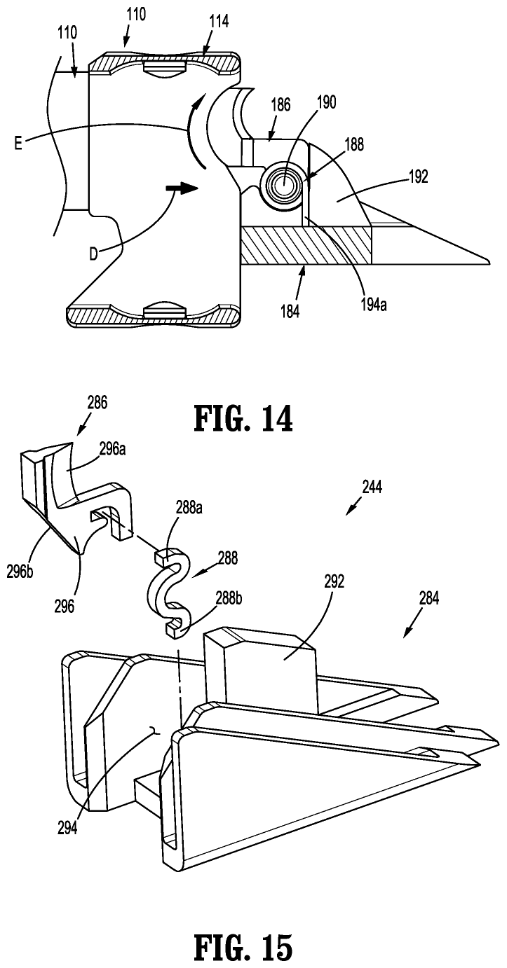

FIGS. 12-14 illustrate an alternative version of the actuation sled assembly 44 shown generally as actuation sled assembly 144. The actuation sled assembly 144 is like the actuation sled assembly 44 (FIG. 5) and includes an actuation sled 184, a knife 186, and a biasing member 188. The actuation sled assembly 144 differs from the actuation sled 44 (FIG. 5) in that the biasing member 88 is replaced with a torsion spring 188 that is supported about a post 190 formed on the central portion 192 of the actuation sled 184. The torsion spring 188 is received about the post 190 on the central portion 192 of the actuation sled 184 and includes one end 194a that is engaged with the central portion 192 of the actuation sled 184 and a second end 194b that is engaged with the knife 186 to urge the knife 186 to the lowered position. As described above regarding the actuation sled assembly 44 (FIG. 5), when the drive assembly 110 is advanced in the direction of arrow “D” in FIG. 14, the working member 114 of the drive assembly 110 engages the knife 186 and pivots the knife 186 in the direction of arrow “E” in FIG. 14 to the raised position (FIG. 14).

FIGS. 15-17 illustrate an alternative version of the actuation sled assemblies described above shown generally as actuation sled assembly 244. The actuation sled assembly 244 is like the actuation sled assemblies 44 (FIG. 5) and 144 (FIG. 12) and includes an actuation sled 284, a knife 286, and a biasing member 288. The actuation sled 284 includes a central portion 292 that defines a recess 294 that receives the knife 286 such that the knife 286 is movable vertically within the recess 294 between raised and lowered positions. The knife 286 includes a body 296 that defines a cutting edge 296a and includes a proximally facing angled surface 296b. The biasing member 288 includes an elastic band that has one end 288a coupled to the knife 286 and a second end 288b coupled to the central portion 292 of the actuation sled 284.

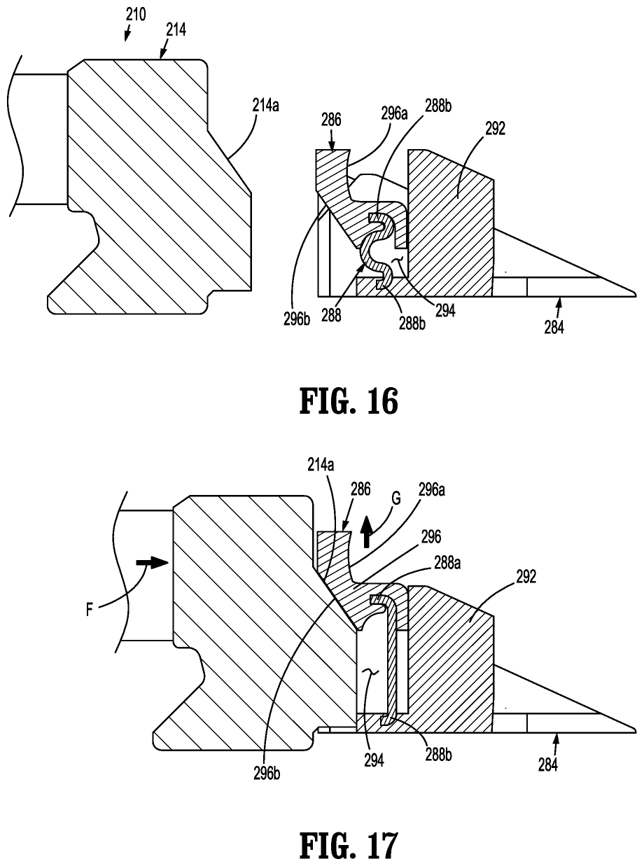

In this version of the stapling device 10, the drive assembly, shown as drive assembly 210, is like drive assembly 110 but includes a working member 214 that has an angled cam surface 214a. When the drive assembly 210 is advanced into engagement with the knife 286 in the direction of arrow “F” in FIG. 17, the angled cam surface 214a of the working member 214 of the drive assembly 210 engages the proximally facing surface 296a of the knife 286 to lift the knife 286 vertically in the direction of arrow “G” in FIG. 17 from the lowered position (FIG. 16) to the raised position (FIG. 17). As the knife 286 moves from the lowered position to the raised position, the biasing member 288 is stretched. When the drive assembly 210 is subsequently retracted after the stapling device 10 (FIG. 1) is fired, the tension in the biasing member 288 returns the knife to the lowered position recessed within the knife slot 52 of the cartridge body 42 of the staple cartridge 28 (FIG. 3).

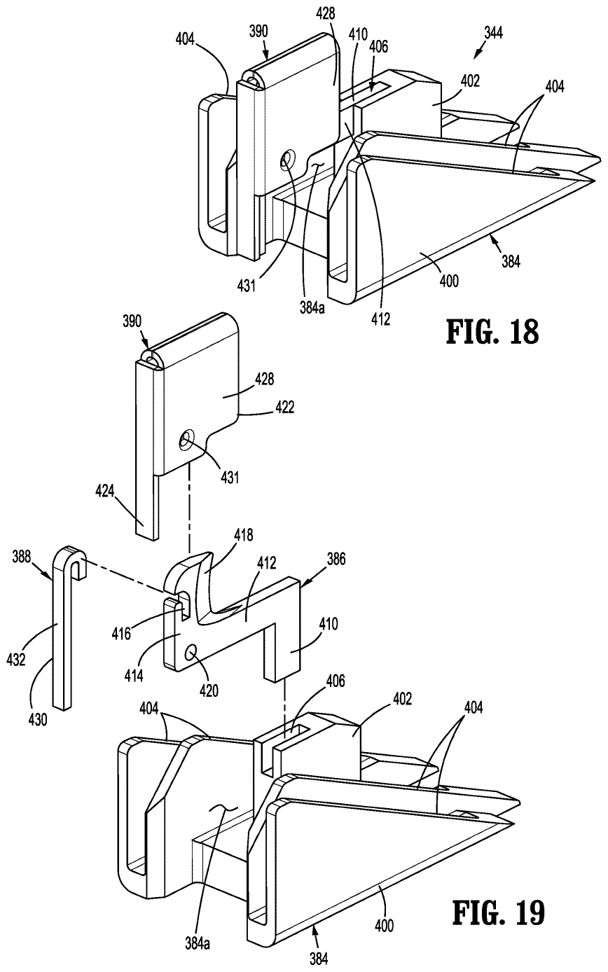

FIGS. 18-22 illustrate an alternative version of the actuation sled assemblies described above shown generally as actuation sled assembly 344. The actuation sled assembly 344 includes an actuation sled 384, a knife 386, a biasing member 388, and a cover 390. The actuation sled 384 is like the actuation sleds described above and includes a sled body 400 that has a central portion 402 and cam surfaces 404 that are positioned on opposite sides of the central portion 402 of the sled body 400. The cam surfaces 404 define ramps that are positioned to engage the pushers 46 (FIG. 3) as the actuation sled 384 moves from the sled retracted position towards the sled advanced position to sequentially eject the staples 48 (FIG. 3) from the cartridge body 42 of the staple cartridge 28. The central portion 402 of the sled body 400 of the actuation sled 384 is received within the knife slot 52 (FIG. 3) of the cartridge body 42 and defines a slot 406. The actuation sled 384 defines a cavity 384a that is open on a proximal side of the actuation sled 384.

The knife 386 includes a base portion 410, a leg 412 that extends proximally from the base portion 410, and a knife portion 414 that extends upwardly from a proximal end of the leg 412. The knife portion 414 defines a recess 416 that opens onto a proximal surface of the knife portion 414 and includes a distally facing cutting edge 418. The base portion 410 of the knife 386 is secured within the slot 406 of the central portion 402 of the actuation sled 400 such that the knife 386 is fixedly secured to the central portion 402 of the actuation sled 400. The knife portion 414 defines concavities or an opening 420.

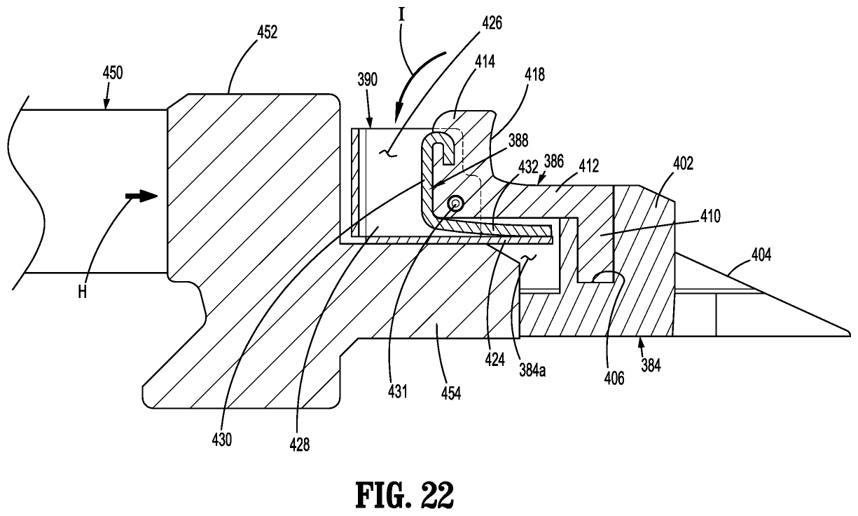

The cover 390 includes a body 422 and a finger 424 that extends downwardly from the body 422. The body 422 defines a pocket 426 (FIG. 22) that is configured to receive the knife portion 414 of the knife 386. The body 422 includes side walls 428 that include protrusions 431 that extend inwardly into the pocket 426 and are received within the opening 420 of the knife 386 to pivotally couple the cover 390 to the knife 386. The cover 390 is pivotable from a first position (FIG. 18) in which the knife portion of the knife 386 is received within the pocket 426 of the cover 390 and the cutting edge 418 is shielded, to a second position in which the cover 390 is spaced from the knife 386 and the cutting edge 418 is exposed.

The biasing member 388 includes a spring arm 430 that is formed from a resilient material, e.g., spring steel, and includes a body 432 having a first end that is coupled to the knife portion 414 of the knife 386. In aspects of the disclosure, the first end of the spring arm 430 has a U-shape and is fixedly received within the recess 416 of the knife portion 414 of the knife 386. Alternately, it is envisioned that the spring arm 430 can be coupled to the knife 386 using a variety of different techniques or configurations. The body 432 of the spring arm 430 in a non-deformed condition extends within the pocket 426 of the cover 390 along a distal surface of the body 432 of the finger 424 of the cover 390 to retain the cover 390 in the first position.

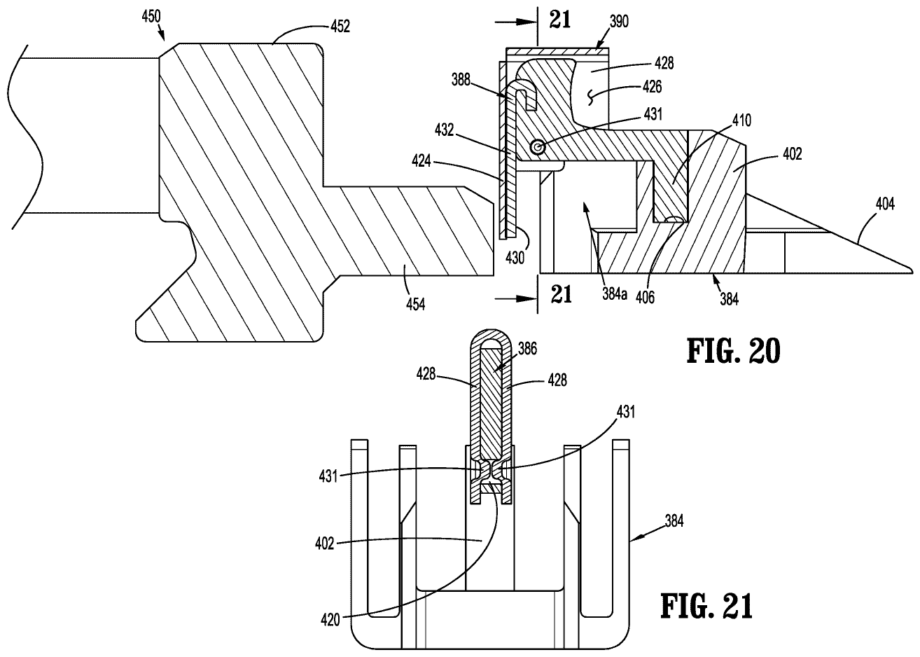

In this version of the stapling device 10, the drive assembly, shown as drive assembly 450 (FIG. 20), is like drive assembly 110 but includes a working member 452 that has an extension 454 that extends distally from a body of the working member 452. When the drive assembly 450 is advanced into engagement with the knife 386 in the direction of arrow “H” in FIG. 22, the extension 454 of the working member 452 of the drive assembly 450 engages the finger 424 of the cover 390 and moves into the cavity 384a of the actuation sled 384 to pivot the cover 390 in the direction of arrow “I” in FIG. 22 from the first position (FIG. 18) to the second position (FIG. 22). As the cover 390 pivots from the first position to the second position, the spring arm 432 is bent inwardly and applies a force on the cover 390 urging the cover 390 back towards the first position. When the drive assembly 450 is subsequently retracted after the stapling device 10 (FIG. 1) is fired, the tension in the spring arm 432 returns the knife 386 to the first position in which the cutting edge 418 of the knife 386 is shielded.

Persons skilled in the art will understand that the devices and methods specifically described herein and illustrated in the accompanying drawings are non-limiting exemplary aspects of the disclosure. It is envisioned that the elements and features illustrated or described in connection with one exemplary aspect of the disclosure may be combined with the elements and features of another without departing from the scope of the disclosure. As well, one skilled in the art will appreciate further features and advantages of the disclosure based on the above-described embodiments. Accordingly, the disclosure is not to be limited by what has been particularly shown and described, except as indicated by the appended claims.

1. A surgical stapling device comprising:
an elongate body having a proximal portion and a distal portion;
a tool assembly supported on the distal portion of the elongate body, the tool assembly including:
an anvil assembly; and
a cartridge assembly coupled to the anvil assembly to facilitate movement of the tool assembly between open and clamped positions, the cartridge assembly including a channel member and a staple cartridge, the channel member defining a channel, the staple cartridge supported within the channel of the channel member and including a cartridge body, staples, pushers, and an actuation sled assembly, the cartridge body having a tissue engaging surface and defining a knife slot and staple receiving slots positioned on each side of the knife slot, the staples received in the staple receiving slots, the actuation sled assembly movable within the cartridge body from a sled retracted position to a sled advanced position to eject the staples from the staple receiving slots of the cartridge body, the actuation sled assembly including an actuation sled, a knife, and a biasing member, the knife received within the knife slot of the cartridge body and including a cutting edge, the knife secured to the actuation sled and movable from a lowered position in which the cutting edge is shielded within the knife slot of the cartridge body to a raised position in which the cutting edge of the knife extends from the knife slot above the tissue engaging surface of the cartridge body, the biasing member secured to the actuation sled and positioned to urge the knife to the lowered position; and
a drive assembly including a working member, the working member positioned proximally of the actuation sled assembly and movable into engagement with the knife of the actuation sled assembly to move the knife from the lowered position to the raised position.

2. The surgical stapling device of claim 1, wherein the actuation sled includes a central portion that is received in the knife slot of the cartridge body, the central portion defining a recess that receives the knife.

3. The surgical stapling device of claim 2, wherein the knife includes a base portion having a circular configuration that is rotatably received within the recess in the central portion of the actuation sled.

4. The surgical stapling device of claim 3, wherein that biasing member is secured to the central portion of the actuation sled and includes a resilient finger that engages the base portion of the knife to urge the knife towards the lowered position.

5. The surgical stapling device of claim 3, wherein that biasing member is secured to the central portion of the actuation sled and includes a torsion spring that is engaged with the knife to urge the knife towards the lowered position.

6. The surgical stapling device of claim 3, wherein the knife includes a curved proximally facing surface and the working end of the drive assembly includes a distally facing curved cam surface, the distally facing curved cam surface of the working end of the drive assembly being movable into the curved proximally facing surface of the knife to move the knife from the lowered position to the raised position.

7. The surgical stapling device of claim 2, wherein the biasing member includes an elastic band.

8. The surgical stapling device of claim 7, wherein the knife is movable vertically within the recess between the lowered position and the raised position.

9. The surgical stapling device of claim 8, wherein the elastic band has a first end secured to the knife and a second end secured to the actuation sled.

10. The surgical stapling device of claim 9, wherein the knife has a proximally facing angled surface and the working member of the drive assembly includes an angled cam surface, the angled cam surface of the working member of the drive assembly movable into engagement with the proximally facing angled surface of the knife to move the knife from the lowered position to the raised position.

11. A cartridge assembly comprising:
a channel member defining a channel; and
a staple cartridge supported within the channel of the channel member and including a cartridge body, staples, pushers, and an actuation sled assembly, the cartridge body having a tissue engaging surface and defining a knife slot and staple receiving slots positioned on each side of the knife slot, the staples received in the staple receiving slots, the actuation sled assembly movable within the cartridge body from a sled retracted position to a sled advanced position to eject the staples from the staple receiving slots of the cartridge body, the actuation sled assembly including an actuation sled, a knife, and a biasing member, the knife received within the knife slot of the cartridge body and including a cutting edge, the knife secured to the actuation sled and movable from a lowered position in which the cutting edge is shielded within the knife slot of the cartridge body to a raised position in which the cutting edge of the knife extends from the knife slot above the tissue engaging surface of the cartridge body, the biasing member secured to the actuation sled and positioned to urge the knife to the lowered position.

12. The cartridge assembly of claim 11, wherein the actuation sled includes a central portion that is received in the knife slot of the cartridge body, the central portion defining a recess that receives the knife.

13. The cartridge assembly of claim 12, wherein the knife includes a base portion having a circular configuration that is rotatably received within the recess in the central portion of the actuation sled.

14. The cartridge assembly of claim 13, wherein that biasing member is secured to the central portion of the actuation sled and includes a resilient finger that engages the base portion of the knife to urge the knife towards the lowered position.

15. The cartridge assembly of claim 13, wherein that biasing member is secured to the central portion of the actuation sled and includes a torsion spring that is engaged with the knife to urge the knife towards the lowered position.

16. (canceled)

17. The cartridge assembly of claim 12, wherein the biasing member includes an elastic band.

18. The cartridge assembly of claim 17, wherein the knife is movable vertically within the recess between the lowered position and the raised position.

19. The cartridge assembly of claim 18, wherein the elastic band has a first end secured to the knife and a second end secured to the actuation sled.

20. (canceled)

21. A cartridge assembly comprising:
a channel member defining a channel; and
a staple cartridge supported within the channel of the channel member and including a cartridge body, staples, pushers, and an actuation sled assembly, the cartridge body having a tissue engaging surface and defining a knife slot and staple receiving slots positioned on each side of the knife slot, the staples received in the staple receiving slots, the actuation sled assembly movable within the cartridge body from a sled retracted position to a sled advanced position to eject the staples from the staple receiving slots of the cartridge body, the actuation sled assembly including an actuation sled, a knife, a cover, and a biasing member, the knife secured to the actuation sled and received within the knife slot of the cartridge body, the knife having a cutting edge, the cover pivotally secured to the knife and defining a pocket, the cover movable from a first position in which the cutting edge of the knife is received within the pocket of the cover and a second position in which the cutting edge of the knife is exposed, the biasing member secured to the knife and positioned to urge the cover towards the first position.

22. The cartridge assembly of claim 21, wherein the biasing member includes a spring arm and the cover includes a finger, the spring arm engaged with the finger of the cover to urge the cover towards the first position.
